(19) United States
(12) Patent Application Publication
(10) Pub. No.: US 2025/0090376 A1
(43) Pub. Date: Mar. 20, 2025

(54) PNEUMATIC FOG PREVENTION FOR OPHTHALMIC LENS

(71) Applicant: Alcon Inc., Fribourg (CH)

(72) Inventors: Michael Sam Cardamone, Keller, TX (US); Paul R. Hallen, Colleyville, TX (US)

(21) Appl. No.: 18/820,453

(22) Filed: Aug. 30, 2024

Related U.S. Application Data
(60) Provisional application No. 63/583,898, filed on Sep. 20, 2023.

(51) Int. Cl. A61F 9/007; A61L 12/00

(52) U.S. Cl. CPC A61F 9/00736; A61L 12/00

(57) ABSTRACT

A lens drying system includes a sterile gas supply, a supply line, and an annular body. The body is connected to the supply line and defines an internal channel. The internal channel, which is in fluid communication with the sterile gas supply via the supply line, defines a plurality of openings. The openings are collectively configured to direct a flow of a sterile gas, e.g., a filtered and desiccated medical grade gas, from the sterile gas supply onto a lens surface of an ophthalmic front lens. This occurs during a non-contact wide-angle visualization process to mitigate fogging of the lens surface. A processor of a surgical console may receive user preference settings, including a desired steady-state flow rate and/or a desired pulse rate of the sterile gas. The processor could then command the desired steady-state flow rate and/or a desired pulse rate from the surgical console.

### INTRODUCTION

The present disclosure relates to automated systems and methods for preventing or removing condensation or “fog” from an ophthalmic lens, e.g., a front lens of an indirect/non-contact wide-angle visualization system.

Vitreoretinal surgery involves the performance of delicate surgical tasks in and around the fundus region of the human eye. Prognosis and diagnosis of injuries, diseases, and other ocular conditions often requires a surgeon to employ a microscope for high-definition visualization. Wide-angle visualization systems use the microscope to provide a magnified real-time view of the fundus region, with optional image capture provided by a digital camera when such images are needed. In this manner, the surgeon is afforded a clear view of the retina, macula, vitreous humor, and surrounding tissues within the eye.

During an ophthalmic visualization procedure, a surgeon might require a wider view of the fundus region than is ordinarily achievable using the microscope's internal lenses. For instance, the surgeon could find it beneficial to view the peripheral retina area when monitoring for retinal tears or detachments. To this end, a wide-angle visualization system uses a specially-constructed ocular lens, which in some implementations is placed directly on the patient's cornea as a contact lens. In indirect/non-contact implementations, an ophthalmic front lens is positioned a short distance away from the patient's cornea surface. The construction of the front lens in either instance provides the desired wide-angle view of the fundus region.

### SUMMARY

Disclosed herein are an indirect/non-contact wide-angle visualization system (WAVS) having a wide-angle ophthalmic front lens and an accompanying method for preventing or removing condensation or “fog” from a surface of the front lens. As appreciated in the art, a front lens of a non-contact WAVS provides the widest possible view of the fundus region when positioned in close proximity to the cornea surface, typically within a few millimeters. As a result of such proximity, however, a patient's normal respiration tends to condense on the lens's inner surface, thus requiring the surgeon or attending operating room staff to periodically lift the front lens for cleaning. To prevent fogging of the front lens, the surgeon might decide to position the front lens a bit farther from the cornea surface. However, doing so reduces the viewing angle. The solutions set forth herein are therefore intended to improve upon the general state of the art of non-contact WAVS by preventing or removing condensation from the front lens, with the present teachings being extendable to other types of ophthalmic lenses.

In accordance with one or more non-limiting exemplary configurations, a lens drying system for use with an ophthalmic front lens of a non-contact WAVS includes a sterile gas supply configured to output a sterile gas, e.g., a filtered and desiccated stream of air, nitrogen, oxygen, or another medical-grade gas. The lens drying system includes a supply line and an annular body. The annular body defines therein an internal channel that is in fluid communication with the sterile gas supply via the supply line. The internal channel defines one or more openings that are collectively configured to direct a flow of the sterile gas onto a lens surface of the front lens to thereby mitigate lens fogging.

In various implementations, the annular body may be constructed from an elastomeric material, a molded plastic, or a metal alloy such as nitinol, and may be configured to connect to or engage with a perimeter surface of the ophthalmic front lens.

The lens drying system could include the ophthalmic front lens, in which case the annular body may be integrally formed within the ophthalmic front lens. The lens drying system could also include a surgical console having a gas port. The sterile gas supply may include a tank containing the sterile gas, with the gas port being connectable to the tank. The surgical console in such a construction is operable for receiving user preference settings from a user of the surgical console, the user preference settings including a desired steady-state flow rate and/or a desired pulse rate of the sterile gas.

Another representative embodiment of the lens drying system includes a supply line connectable to a sterile gas supply, an ophthalmic front lens, and an annular body defining an internal channel therein in fluid communication with a sterile gas supply via the supply line. The internal channel defines openings arranged on a perimeter surface of the annular body, the openings being collectively configured to direct a flow of a desiccated inert gas from the internal channel and onto a lens surface of the ophthalmic front lens to mitigate fogging of the lens surface. The annular body is connectable to or formed integrally with the ophthalmic front lens.

Also disclosed herein is a method for use with an ophthalmic front lens. An exemplary embodiment of the method includes providing an annular body defining an internal channel therein. The internal channel defines a plurality of openings through a diameter surface of the annular body. The method may include directing a flow of sterile gas from a sterile gas supply to the internal channel of the annular body via a supply line, and also directing the flow of the sterile gas through the openings and onto a lens surface of the ophthalmic front lens to thereby mitigate fogging of the lens surface.

The method could optionally include receiving user preference settings via a processor of a surgical console, the user preference settings including a desired steady-state flow rate and/or a desired pulse rate of the sterile gas. Such an implementation might also include outputting the desired steady-state flow rate and/or a desired pulse rate from the surgical console in accordance with the user preference settings.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 illustrates an exemplary ophthalmic surgical suite equipped with a non-contact wide-angle visualization system (WAVS) having a front lens, condensation on which is removeable or preventable in accordance with the present disclosure.

FIG. 2 is an illustration of an embodiment of the front lens shown in FIG. 1.

FIG. 3 is a perspective view illustration of the front lens of FIGS. 1 and 2 according to a representative embodiment.

FIG. 4 is a plan view illustration of the front lens of FIGS. 1 and 2 according to another representative embodiment.

FIG. 5 is a flow chart describing an exemplary embodiment of a method for preventing or removing condensation from the front lens of FIGS. 1-4.

FIG. 6 is a perspective view illustration of an alternative attachable configuration of a lens drying system located external to the front lens.

FIG. 7 is a cross-sectional illustration of flexible tubing usable as part of the lens drying system of FIG. 6.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

Embodiments of the present disclosure are described in detail herein. Disclosed embodiments are provided as examples, with other embodiments possibly taking alternative forms. The Figures are not necessarily drawn to scale. For instance, some features could be exaggerated or minimized to show details of particular components. Therefore, specific structural and functional details disclosed herein are not to be interpreted as limiting, but merely as a representative basis for teaching one skilled in the art to employ the present disclosure.

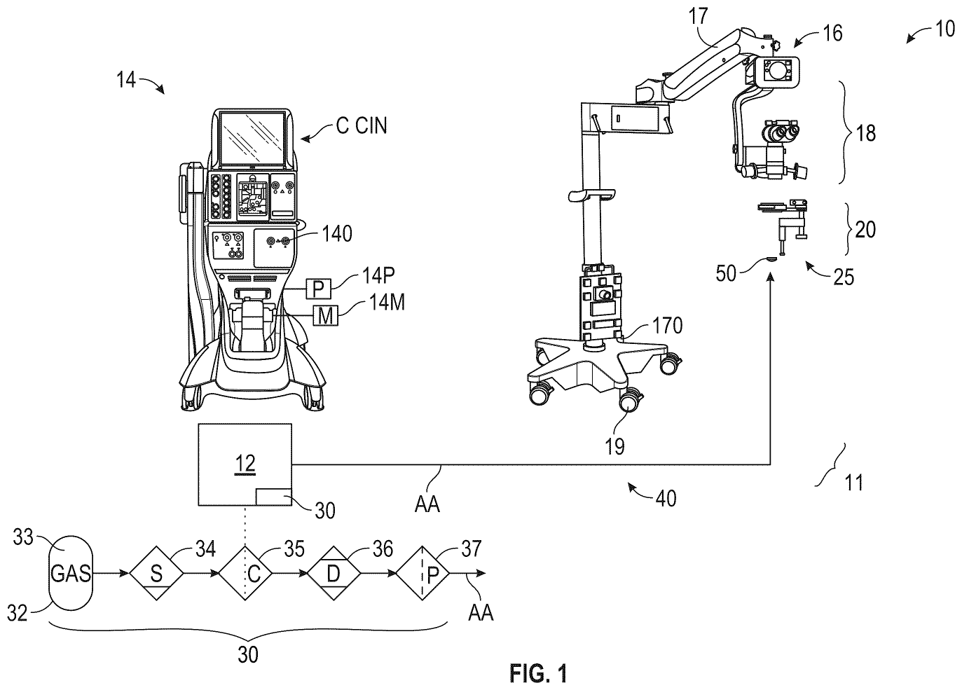

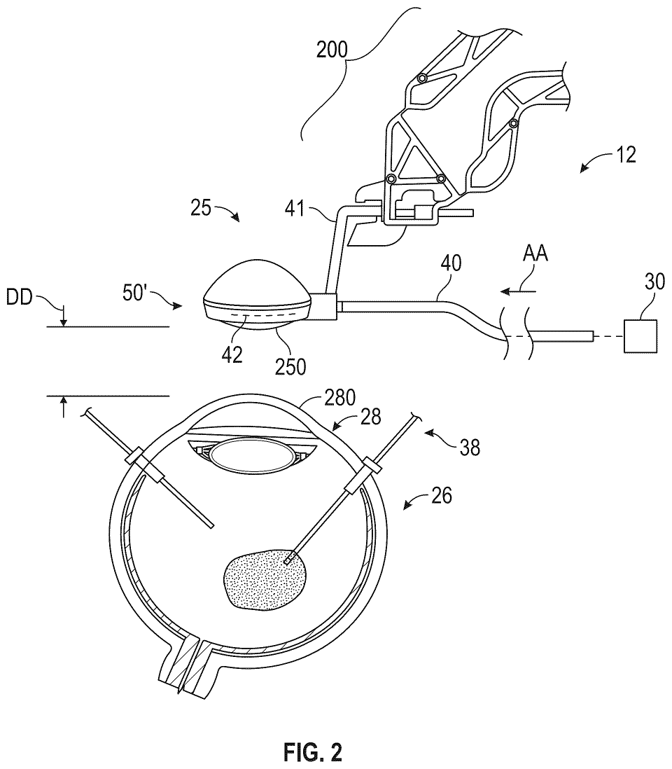

Referring to the drawings, wherein like reference numbers refer to like components, and beginning with FIG. 1, a visualization system 10 includes a lens drying system 11 in accordance with the disclosure. The lens drying system 11 in turn includes a fluidic system 12, a surgical console 14, and a wide-angle viewing system (WAVS) 16. The WAVS 16 for its part includes a serial robot arm 17 operatively connected to a base 170, with the base 170 as shown being equipped with a set of wheels 19. The serial robot arm 17 is also connected to an ophthalmic microscope 18. In the illustrated configuration, the microscope 18 is connected to or equipped with a wide-angle lens assembly 20, e.g., the commercially available ZEISS Resight™ Fundus Imaging System or OCULUS BIOM®, modified to include an ophthalmic front lens 25 as described below with particular reference to FIGS. 2, 3, and 4.

As noted above, an ocular lens of an indirect/non-contact WAVS provides the widest possible view of the fundus region of a patient's eye when such a lens is positioned in close proximity to the cornea surface. Referring briefly to FIG. 2, for instance, a patient's eye 26 is depicted undergoing a vitrectomy surgery. During vitrectomy surgery, the front lens 25 described herein is positioned at a standoff distance (DD) away from a surface 280 of the cornea 28, with the standoff distance (DD) being on the order of, e.g., 5-10 millimeters (mm). Absent provision of filtered and desiccated inert gas flow (AA) from the fluidic system 12, the patient's warm breath would tend to condense on a lower surface 250 of the front lens 25, i.e., the particular surface located closest to the cornea surface 280. This would require the surgeon or attending medical staff to increase the standoff distance (DD) in an attempt at reducing fogging of the front lens 25, or to periodically wipe the lower surface 250 to clear any accumulated condensation or lens fog.

Referring again to FIG. 1, the fluidic system 12 in accordance with an embodiment of the disclosure includes a sterile gas supply 30 configured to output the filtered and desiccated inert gas flow (AA). The fluidic system 12 also include a supply line 40, e.g., flexible tubing or hose, and an annular body 50, the latter of which is also shown in FIGS. 2-4 and described below. As the filtered and desiccated inert gas flow (AA) is intended to prevent fogging of the front lens 25, thereby making the front lens 25 effectively “non-fogging” in accordance with the present disclosure, the sterile gas supply 30 as contemplated herein must be sufficiently dry.

Due to use of the front lens 25 in close proximity to the patient's eye 26 of FIG. 2, the filtered and desiccated inert gas flow (AA) must also be sufficiently devoid of suspended particulate matter and oil. To that end, the sterile gas supply 30 in a possible implementation includes a pressure-regulated tank 32 containing an application-suitable sterile form of an inert gas 33, e.g., medical air, nitrogen, or oxygen. The inert gas 33 may include prefiltered air/gas supply that is devoid of moisture or otherwise sufficiently desiccated. In such an embodiment, the tank 32 may contain the filtered and desiccated gas flow (AA), delivering metered and pressure-regulated amounts of the inert gas 33 to the front lens 25 via the supply line 40 with or without downstream filtration.

Alternatively, the sterile gas supply 30 of FIG. 1 could include several stages of drying and filtration situated downstream of the tank 32. In the illustrated exemplary setup, the inert gas 33 could pass from the tank 32 through an air-water separator(S) stage 34 to remove entrained liquid water, with the inert gas 33 thereafter passing through a coalescing filter (C) 35. As appreciated in the art, suitable coalescing filters typically are constructed from borosilicate glass fibers configured to remove submicronic particulate matter and oil/water droplets. A drying stage (D) 36, such as a bed of disposable or regeneratable hydroscopic desiccant beads, may be positioned in line with the coalescing filter 35 to help reduce the dewpoint of the inert gas 33. A particulate filter (P) 37 such as pleated cellulose or sintered plastic may be situated downstream of the drying stage 36 to prevent propagation of desiccant beads or dust to the point of use.

In the simplified illustration of FIG. 1, the surgical console 14 also includes a gas port 140 configured to connect to the tank 32. As appreciated in the art, ophthalmic surgical consoles such as the surgical console 14 are typically equipped with multiple columns and rows of connection ports to provide the necessary electrical, data, pressure, irrigation, suction, and other power needed for supporting a given surgical procedure and its related surgical tools 38 (see FIG. 2). One such connection port could be configured herein as the gas port 140 within the scope of the disclosure, with the gas port 140 constructed to receive an end of the supply line 40.

Still referring to FIG. 1, the surgical console 14 may be configured as or include an electronic control unit in communication with other systems or components, e.g., via a wired or wireless communications network or individual transfer conductors. The surgical console 14 could include one or more processors (P) 14P and sufficient computer-readable storage media/tangible-not transitory memory (M) 14M, e.g., optical, magnetic, flash, or other types of read only memory, along with application-sufficient amounts of random-access memory, electrically-erasable programmable read only memory, etc. The processor(s) for their part may be constructed from various combinations of Application Specific Integrated Circuit(s) (ASICs), Field-Programmable Gate Arrays (FPGAs), electronic circuits, central processing units, microprocessors, and the like.

In some configurations, the surgical console 14 may be operable for receiving user preference settings (CCI) from a surgeon, staff, or another user of the surgical console 14. The user preference settings (CCIN) may include settings for the filtered and desiccated inert gas flow (AA), along other possible control parameters as noted above. For instance, a surgeon performing an ophthalmic procedure on the eye 26 of FIG. 2 may prefer to address potential fogging via a continuous steady-state flow of the filtered and desiccated inert gas flow (AA) across the lower surface 250 of the front lens 25. Alternatively, the surgeon could prefer to use an intermittent or pulsed flow, the latter of which might dry the eye 26 a bit less than would a constant/steady-state flow. Thus, the user preference settings (CCI) in one or more embodiments may include a desired steady-state flow rate and/or a desired pulse rate of the filtered and desiccated inert gas flow (AA).

Referring once again to FIG. 2, the annular body 50 of the present disclosure is connected to or formed integrally with the front lens 25. In the illustrated embodiment, the annular body 50 is connected to the wide-angle lens assembly 20 of FIG. 1 via a connection piece 200, e.g., a multi-armed flexible member, which in turn is connected to an arm segment 41. The annular body 50 and possibly the arm segment 41 could define therein an internal channel 42, itself rung-shaped/annular, that is in fluid communication with the sterile gas supply 30 via the supply line 40. The internal channel 42 receives the filtered and desiccated inert gas flow (AA) and thereafter directs the same onto the lower surface 250 of the front lens 25. In this manner, the present approach is able to mitigate problems associated with fogging of the lower surface 250.

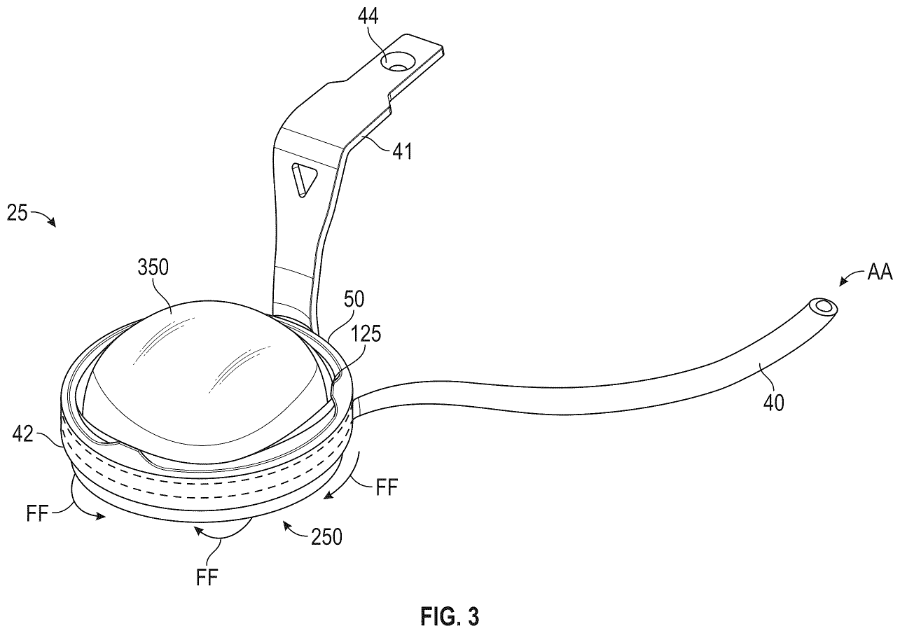

The front lens 25 and the annular body 50 are depicted in perspective view in FIG. 3 according to an exemplary configuration. The arm segment 41 may include a distal end 44 that is configured to engage with/be supported by the connection piece 200. Similar to FIG. 2, the annular body 50 is formed as a complete annulus or ring that fully circumscribes the front lens 25, i.e., by surrounding a perimeter surface thereof. In this embodiment, an upper surface 350 of the front lens 25 is oriented toward the microscope 18 of FIG. 1, with the lower surface 250 positioned in close proximity to the eye 26 (FIG. 2) and thus prone to fogging as noted above.

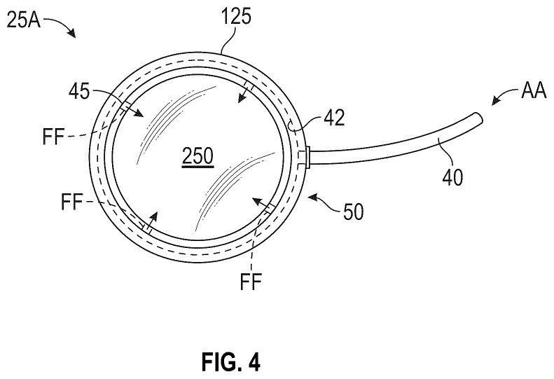

The internal channel 42 defines a plurality of openings 45 (see FIG. 4) collectively configured to direct a flow of the filtered and desiccated inert gas flow (AA) onto the lower surface 250. In different embodiments, each of the openings 45 could be circular in cross-section, or possibly oval, rectangular, or formed as a slot. The openings 45 may be equally or unequally spaced around the circumference of the annular body 50 to provide optimal flow across the lower surface 250.

Further with respect to the annular body 50, this component may be constructed from a wide range of application-suitable sterile or sterilizable materials. By way of a few illustrative examples, the annular body 50 could be constructed of a metal such as stainless steel or aluminum, a metal alloy such as but not limited to nitinol, or a molded plastic or elastomeric material such as an application-suitable polymer, PVC (Polyvinyl Chloride), or a flexible shape-memory alloy (SMA). The annular body 50 could be configured to connect to or support a perimeter surface 125 of the front lens 25, e.g., via a set of radial tabs or other suitable detents.

Referring to FIG. 4, in yet another construction the annular body 50 could be integrally formed with the front lens 25. Such a construction is illustrated in FIG. 4 as an integral front lens 25A. For instance, the integral front lens 25A may be formed from molded plastic to define the above-described openings 45. Although four equally-spaced opening 45 are depicted, more or fewer openings 45 may be used in other configurations, with such openings 45 not necessarily being equally-spaced as noted above. In operation, the integral front lens 25A, as well as the separate body 50 and lens 25 of FIG. 3, the inert gas flow (AA) is directed via the supply line 40 and into the internal channel 42. The inert gas flow (AA) then circulates within the internal channel 42. Upon reaching the openings 45, the filtered and desiccated inert gas flow (AA) escapes from the annular body 50 therethrough and passes as directed gas flow (FF) over the lower surface 250. The relative dryness and flow of the directed gas flow (FF) thereafter removes accumulated fogging from the lower surface 250 of the integral front lens 25A.

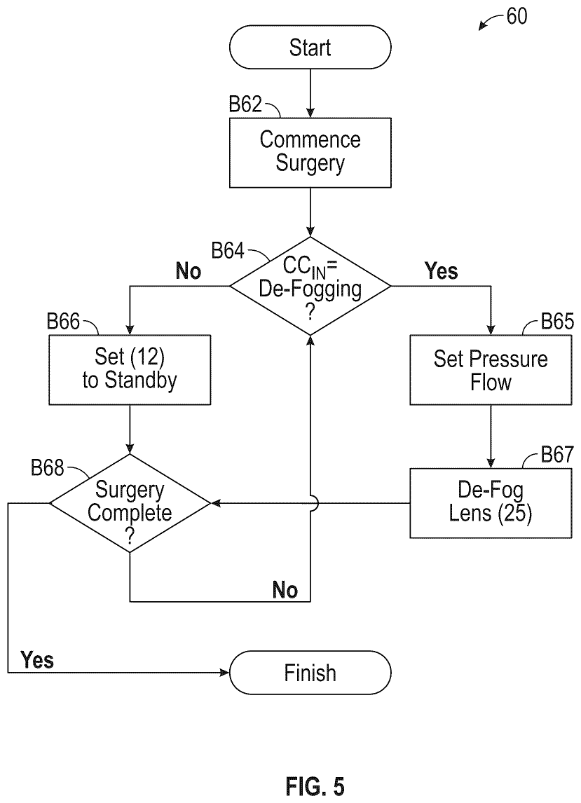

Referring to FIG. 5, a method 60 is described in terms of discrete process steps, segments, or logic blocks for clarity. Some of the hardware solutions set forth above may be implemented in software, for example by programming the memory 14M of the surgical console 14 shown in FIG. 1. Thus, a corresponding automated routine may initialize (“Start”) with commencement at block B62 of a vitrectomy or other surgical procedure of the eye 26 show in FIG. 2. The method 60 then proceeds to block B64.

At block B64, the surgeon may select de-fogging of the front lens 25 or 25A. For example, the surgeon may input or command the user preference settings (CCIN), e.g., via the surgical control console 14 as described above. Thus, block B64 may entail determining, via the processor 14P of such a surgical console 14, whether the surgeon desires de-fogging operation of the front lens 25 or the integral front lens 25A, for instance by processing the preference settings (CCIN). The method 60 proceeds to block B65 when de-fogging is desired, with the method 60 otherwise proceeding to block B66.

At block B65, the processor 14P of the surgical console 14 may set the pressure and/or flow rate of the filtered and desiccated inert gas (AA). For example, the user preference settings (CCIN) could request a desired steady-state flow rate and/or a desired pulse rate of the inert gas 33 of FIG. 2. In response, the processor 14P could command associated flow control hardware, e.g., valves, pressure and/or flow regulators, etc., to output the filtered and desiccated inert gas (AA) at a pressure and flow rate corresponding to the user preference settings (CCIN). The method 60 then proceeds to block B67.

Block B66, which may be reached from block B64 absent the user preference settings (CCIN), includes setting the fluidic system 12 to a “standby” mode. In such a mode, the fluidic system 12 remains available to provide the filtered and desiccated inert gas (AA) if and when it might be needed during surgery. The method 60 thereafter proceeds to block B68.

At block B68, the processor 14P of the surgical console 14 may determine whether the surgical procedure than began at block B62 is complete. The method 60 proceeds to block B64 when the surgery is ongoing, and to “Finish” when the surgery is complete. At “Finish”, the surgeon would no longer require the front lens 25 or 25A, and thus the method 60 would start anew at block B62 for a subsequent surgery.

Block B67 of FIG. 5 includes de-fogging the front lens 25 using the filtered and desiccated inert gas (AA), with the filtered and desiccated inert gas (AA) expelled from the annular body 50 at the desired steady-state flow rate or pulse rate. The method 60 thereafter proceeds to block B68.

The present solutions thus envision disposing the annular body 50 of FIGS. 1-4 around a circumference of a wide-angle lens, i.e., the front lens 25 or 25A, which in turn is held a short distance above the eye 26 for viewing via the microscope 18. The annular body 50 thus acts as a perforated lens holder that is sized to fit a specific front lens 25 or 25A. In some implementations, the supply line 40 and the annular body 50 could be provided as a single-use kit, either as a universal kit suitable for most configurations of the front lens 25 or 25A, or as a variety of sizes and shapes to fit the most common commercially-available constructions thereof. In other implementations the annular body 50 could be autoclavable or otherwise sterilizable, and thus re-usable. These and other attendant benefits of the present non-fogging solutions to indirect/non-contact wide-angle visualization will be readily appreciated by those skilled in the art in view of the foregoing disclosure.

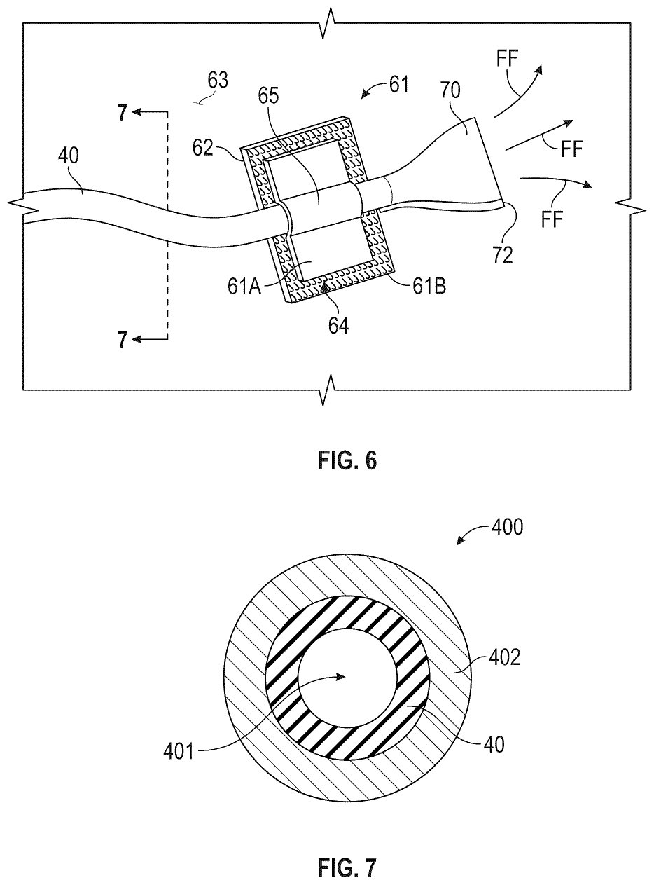

Referring now to FIG. 6, a lens drying system 11A may be used to achieve similar fog-removing or fog-preventing ends when full or partial integration of lens drying structure with the front lens 25 of FIG. 3 is not desired or practicable. The lens drying system 11A according to one or more embodiments includes the supply line 40, a sterile pad 61, and a diffuser body 70 disposed at a distal end of the supply line 40 in proximity to the front lens 25. For example, a surgeon or medical staff could adhere the sterile pad 61 to a likewise sterile surgical draping 63 covering the patient. In the illustrated example, the sterile pad 61 could include a hook-and-loop pad set, e.g., VELCRO®, having a cover pad 61A and a base pad 61B. The base pad 61B could have an adhesive surface 62 configured to adhere or stick to the surgical draping 63 at a desired location. While one sterile pad 61 is illustrated for simplicity, multiple sterile pads 61 could be used along the length of the supply line 40 as desired.

The cover pad 61A for its part may include an arcuate bridge portion 65 through which the supply line 40 is routed, such that the supply line 40 is securely trapped between the cover pad 61A and the base pad 61B when the cover pad 61A and base pad 61B are pressed together. While the base pad 61B is depicted with loops 64 facing the cover pad 61A, with the cover pad 61A having the mating hooks, the opposite construction may be used in this implementation, i.e., with the cover pad 61A and base pad 61B having loops and hooks, respectively. Other implementations can be readily conceived of that would achieve the same positioning ends, including the use of removable adhesives in lieu of hook-and-loop.

The diffuser body 70 of FIG. 6 may have an outwardly-tapered or flattened cone-shaped profile to help diffuse and form the directed gas flow (FF) upon its discharge from the supply line 40. That is, the outer diameter of the supply line 40 could gradually widen toward an exhaust port located at a distal end 72 of the diffuser body 70. Rather than directing the filtered and desiccated inert gas flow (AA) through a small-diameter center opening 401 of the supply line 40 (see FIG. 7), therefore, the diffuser body 70 may be constructed to spread the gas flow (AA) as the directed gas flow (FF) over more surface area of the proximate front lens 25 of FIG. 1.

Referring briefly to FIG. 7, the supply line 40 of FIG. 6 could be alternatively constructed as a shape-memory supply line 400 in one or more configurations. As medical tubing tends not to hold its curvature when positioned, thus potentially complicating implementation of the solution shown in FIG. 6, a possible solution is to insert the supply line 40 or segments thereof through a shape-memory sleeve 402, e.g., a shape-memory alloy (SMA) tube or a bendable plastic tube. When the shape-memory supply line 400 is positioned relative to the surgical draping 63 of FIG. 6, therefore, the use of the shape-memory sleeve 402 on curves or bends in the supply line 40 may allow the shape-memory supply line 400 to remain relatively immobile.

As those of ordinary skill in the art will understand, various features illustrated and described with reference to any one of the Figures can be combined with features illustrated in one or more other Figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Embodiments shown in the drawings or the characteristics of various embodiments mentioned in the present description are not necessarily to be understood as being independent of each other. It is possible that each of the characteristics described in a given embodiment could be combined with one or more other desired characteristics from other embodiments, resulting in other embodiments not described in words or by reference to the drawings. Accordingly, such other embodiments fall within the framework of the scope of the appended claims.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

What is claimed is:

1. A lens drying system, comprising:
a sterile gas supply;
a supply line; and
annular body connected to the supply line and defining an internal channel, wherein the internal channel is in fluid communication with the sterile gas supply via the supply line, and wherein the internal channel defines a plurality of openings collectively configured to direct a flow of a sterile gas from the sterile gas supply onto a lens surface of an ophthalmic front lens during a non-contact wide-angle visualization process to thereby mitigate fogging of the lens surface.

2. The lens drying system of claim 1, wherein the annular body is constructed from an elastomeric material.

3. The lens drying system of claim 1, wherein the annular body is constructed from a metal alloy.

4. The lens drying system of claim 3, wherein the metal alloy includes nitinol.

5. The lens drying system of claim 1, wherein the annular body is configured to connect to or engage with a perimeter surface of the ophthalmic front lens.

6. The lens drying system of claim 1, further comprising:
the ophthalmic front lens.

7. The lens drying system of claim 6, wherein the annular body is integrally formed within the ophthalmic front lens.

8. The lens drying system of claim 7, wherein the annular body is constructed of molded plastic.

9. The lens drying system of claim 1, further comprising:
a surgical console having a gas port, wherein the sterile gas supply includes a tank containing the sterile gas, and wherein the gas port is configured to connect to the tank.

10. The lens drying system of claim 9, wherein the surgical console is operable for receiving user preference settings from a user of the surgical console, the user preference settings including a desired steady-state flow rate and/or a desired pulse rate of the sterile gas.

11. A lens drying system, comprising:
a supply line connectable to a sterile gas supply;
an ophthalmic front lens; and
an annular body defining an internal channel therein in fluid communication with a sterile gas supply via the supply line, wherein:
the internal channel defines openings arranged on a perimeter surface of the annular body, the openings being collectively configured to direct a flow of a desiccated inert gas from the internal channel and onto a lens surface of the ophthalmic front lens to mitigate fogging of the lens surface; and
the annular body is connectable to or formed integrally with the ophthalmic front lens.

12. The lens drying of claim 11, wherein the annular body is constructed of a molded plastic.

13. The system of claim 12, wherein the annular body is integrally formed with the ophthalmic front lens from the molded plastic.

14. A method for use with an ophthalmic front lens, comprising:
providing an annular body defining an internal channel therein, wherein the internal channel defines a plurality of openings through a diameter surface of the annular body;
directing a flow of sterile gas from a sterile gas supply to the internal channel of the annular body via a supply line; and
directing the flow of the sterile gas through the openings and onto a lens surface of the ophthalmic front lens to thereby mitigate fogging of the lens surface.

15. The method of claim 14, further comprising:
receiving user preference settings via a processor of a surgical console, the user preference settings including a desired steady-state flow rate and/or a desired pulse rate of the sterile gas; and
outputting the desired steady-state flow rate and/or a desired pulse rate from the surgical console in accordance with the user preference settings.
